(19) United States
(12) Patent Application Publication
(10) Pub. No.: US 2016/0279437 A1
(43) Pub. Date: Sep. 29, 2016

(54) LED PAD, METHOD FOR MANUFACTURING THE SAME AND PERSONAL TREATMENT APPARATUS COMPRISING THE SAME

(71) Applicant: PEACHTECH CO., LTD., Seoul (KR)

(72) Inventor: Hae Sung PARK, Seoul (KR)

(73) Assignee: PEACHTECH CO., LTD., Seoul (KR)

(21) Appl. No.: 15/034,182

(22) PCT Filed: Nov. 5, 2014
(86) PCT No.: PCT/KR2014/010541

§ 371 Date: May 4, 2016

(30) Foreign Application Priority Data
Nov. 12, 2013 (KR) 10-2013-0136868
Aug. 27, 2014 (KR) 10-2014-0112250

(51) Int. Cl. A61N 5/06

(52) U.S. Cl. CPC A61N 5/06; A61N 2005/0659; A61N 2005/0652

(57) ABSTRACT

Disclosed are an LED pad, a method for manufacturing the LED pad, and a personal treatment apparatus comprising the LED pad which comprises a lower silicone case, an upper silicone case coupled to the lower silicone case, and an LED module which comprises a plurality of LEDs and a flexible printed circuit board mounted with the plurality of LEDs and which is located inside a silicone case configured by combination of the lower silicone case and the upper silicone case.

### TECHNICAL FIELD

The present invention relates to an LED pad, a method for manufacturing the LED pad, and a personal treatment apparatus including the LED pad. More particularly, the present invention relates to an LED pad, a method for manufacturing the LED pad and a personal treatment apparatus including the LED pad, for maximizing the efficient use of LED light and the effect of light therapy by employing a heat release structure which prevents contaminations due to exposure to the outside, provides a flexible LED pad to make a close contact with user's body and minimizes heat released to user's body in close contact therewith.

### BACKGROUND ART

Typically, NIR (Near Infrared ray) (for example, light of which the wavelength ranges between 700 nm and 3000 nm) within infrared ray is known for its various effects. It is known that such NIR forms nitric oxide in user's body and is thus able to expand or create blood vessels, create collagen or relieve pain when the user's body, for example, skin, is exposed to NIR.

Based on the aforementioned effects, personal treatment apparatuses by using LEDs (Light Emitting Diodes) emitting NIR are generally known. The personal treatment apparatuses are composed of a plurality of NIR LEDs, a substrate mounted with the plurality of NIR LEDs and a case housing the NIR LEDs and the substrate and insulating from the outside.

Conventional personal treatment apparatuses have various issues involved in being used for general individual user.

First, conventional personal treatment apparatuses are structured to protect internal components by using a hard case to protect a plurality of NIR LEDs and a PCB mounted with the plurality of NIR LEDs from exposure to the outside. Therefore, the personal treatment apparatuses do not maximize the effect of NIR because they do not make a close contact with a specific portion of user's body (for example, forearms or knees, etc.).

In addition, although some personal treatment apparatuses use transparent silicone to allow NIR LED light to be transmitted, the transparent silicone is used as a filling material for protecting the PCB, or the NIR LEDs. As a result, internal components may not be completely insulated from external impurities or moisture to result in defects, or exposure thereof to external impurity sources causes the personal treatment apparatuses not to function normally as intended.

Meanwhile, the NIR LEDs of the conventional personal treatment apparatuses emit light to generate much heat of which the highest temperature is measured at the locations where the NIR LEDs are mounted, but relatively low temperature is measured as it is farther from the location of each NIR LED.

Temperature change in the aforementioned personal treatment apparatuses suggests that they do not make a close contact completely with the skin of user's body for treatment. That is, it is not allowed to increase NIR LED light output to avoid burns caused by high temperature at a local point of specific surface of the personal treatment apparatuses. Accordingly, intended treatment or the effect of the conventional personal apparatuses is not fully implemented.

In addition, if transparent silicone is used for the personal treatment apparatuses, it is essential to consider the heat generated in the NIR LEDs. In general, in a personal treatment apparatus which uses NIR LEDs, approximately 30% of power is consumed through LED light, and the remaining approximately 70% thereof is released as heat.

In particular, a personal treatment apparatus by using transparent silicone to make a close contact with user's body requires a release structure effective for releasing heat. Because the silicone itself which makes a close contact with user's body accumulates the heat released by the LEDs, users may experience extreme burning sensation on or in their skin or have burns. Therefore, it is not allowed to increase the amount of NIR LED light to lower the effect of light therapy.

Therefore, there is a need for an LED pad, a method for manufacturing the LED pad, and a personal treatment apparatus including the LED pad to solve the aforementioned problems of conventional personal treatment apparatuses.

### DISCLOSURE

### Technical Problem

Therefore, the present invention devised to solve the aforementioned problems provides an LED pad, a method for manufacturing the LED pad and a personal treatment apparatus including the LED pad, by making the LED pad flexible to enable a close contact easily with user's body.

Further, the present invention provides an LED pad, a method for manufacturing the LED pad and a personal treatment apparatus including the LED pad, to prevent the LED pad from being exposed to external impurities or moisture, and further prevent short circuits of the PCB in the LED pad or the circuit in the PCB.

In addition, the present invention provides an LED pad, a method for manufacturing the LED pad and a personal treatment apparatus including the LED pad, to integrate the case for housing NIR LEDs as one unit by using silicone to avoid easy disassembly and make the LED pad highly flexible.

In addition, the present invention provides an LED pad, a method for manufacturing the LED pad and a personal treatment apparatus including the LED pad, to disperse the heat radiated from NIR LEDs to keep the LED pad at constant temperature in order to prevent user's skin burns which are caused by a close contract with the LED pad, and increase NIR LED light output.

In addition, the present invention provides an LED pad, a method for manufacturing the LED pad and a personal treatment apparatus including the LED pad, to eliminate the heat radiated from NIR LEDs from the skin side which makes a close contact with the pad to lower the level of burning sensation the LED pad user experiences and increase light output to enhance the effect of light therapy.

It should be noted that the technical effect of the present invention is not limited to those mentioned above, and other technical effects not mentioned above will be apparent to those skilled in the art of the present invention from the following description.

### Technical Solution

In accordance with an embodiment of the present invention, there is provided an LED pad including: a lower silicone case; an upper silicon case integrated with the lower silicone case; and an LED module comprising a plurality of LEDs and a flexible PCB mounted with the plurality of LEDs and positioned in a silicone case formed by integrating the lower silicone case with the upper silicone case.

In the embodiment, the flexible PCB of the LED module includes: a reflective layer configured to reflect incident light through the upper silicone case and being positioned on the surface of the flexible PCB; and a heat conductance layer configured to disperse heat radiated by the plurality of LEDs and formed on the surface opposite to the surface where the plurality of LEDs are mounted.

In the embodiment, the LED module further includes a power connector configured to supply power to the plurality of LEDs and being mounted on the flexible PCB, and the power connector supplies power to the plurality of LEDs through a PCB pattern formed in the heat conductance layer and is mounted to protrude as far as the thickness of the edge surface of the lower silicone case.

In the embodiment, the pad further includes one or more adhesive pads integrated with the lower silicone case and for fixing the LED pad, and the upper silicone case being transparent is integrated chemically with the lower silicone case along the edge of the lower silicone case, the heat conductance layer is copper foil, and the plurality of LEDs are LEDs emitting Near-Infrared Ray.

In the embodiment, the upper silicone case comprises grooves positioned between one LED and other LEDs adjacent to the one LED.

In the embodiment, each of the plurality of LEDs is positioned on the flexible PCB to be spaced apart from other LEDs adjacent to itself in one direction at defined intervals, and other LEDs adjacent to itself in a different direction from the one direction at defined intervals, and the grooves of the upper silicone case are those positioned only at the one direction or those positioned at the one direction and the different direction.

In the embodiment, the distance from the flexible PCB to the surface of the upper silicone case is configured to be longer than the distance from the flexible PCB to the surface of the lower silicone case, and the LED pad releases heat radiated by the plurality of LEDs to the lower silicone case through the heat conductance layer.

In accordance with another embodiment of the present invention, there is provided a method for manufacturing an LED pad, the method including: (a) mounting an LED module comprising a plurality of LEDs and a flexible PCB mounted with the plurality of LEDs; and (b) molding an upper silicone case to be integrated with a lower silicone case with transparent liquid silicone along the edge of the lower silicone case.

In the embodiment, the LED module further includes a power connector supplying power to the plurality of LEDs, being mounted on the flexible PCB and protruding as far as the edge thickness from the flexible PCB, and the method for manufacturing an LED pad further includes: adding a material to a power inlet of the power connector for preventing the transparent liquid silicone from entering the power inlet before said (a) mounting an LED module; and removing the added material after said (b) molding an upper silicone case.

In accordance with further another embodiment of the present invention, there is provided a personal treatment apparatus comprising an LED pad, the apparatus including: an LED pad; and a controller for controlling power supplied to a plurality of LEDs of the LED pad, wherein the LED pad includes a lower silicone case; an upper silicon case integrated with the lower silicone case; and an LED module comprising a plurality of LEDs and a flexible PCB mounted with the plurality of LEDs and positioned in a silicone case formed by integrating the lower silicone case with the upper silicone case.

### ADVANTAGEOUS EFFECTS

As described above, in accordance with the present invention, the LED pad, the method for manufacturing the LED pad and the personal treatment apparatus including the LED pad implement a flexible LED pad to enable it to make a close contact easily with user's body.

In addition, in accordance with the present invention, the LED pad, the method for manufacturing the LED pad and the personal treatment apparatus including the LED pad implement exposure to external impurities or moisture to be prevented, and further short circuits of the substrate in the LED pad or the circuit in the substrate to be prevented.

In addition, in accordance with the present invention, the LED pad, the method for manufacturing the LED pad and the personal treatment apparatus including the LED pad implement the case for housing the NIR LEDs to be integrated as one unit by using silicone to avoid easy disassembly and make the LED pad highly flexible.

In addition, in accordance with the present invention, the LED pad, the method for manufacturing the LED pad and the personal treatment apparatus including the LED pad disperse heat radiated from the NIR LEDs to keep the LED pad at constant temperature in order to prevent burns of user's skin which could be caused by a close contact with the LED pad, and increase the light output of the NIR LEDs.

In addition, in accordance with the present invention, the LED pad, the method for manufacturing the LED pad and the personal treatment apparatus including the LED pad allow the heat radiated from the NIR LEDs to be eliminated from the skin side which makes a close contact with the LED pad, in order to lower the level of burning sensation its user experiences and increase light output to enhance the effect of light therapy.

It should be noted that the effect of the present invention is not limited to those mentioned above, and other effects not mentioned above will be apparent to those skilled in the art of the present invention from the following description.

### BRIEF DESCRIPTION OF DRAWINGS

FIG. 1 is an exemplary configuration diagram of a personal treatment apparatus;

FIG. 2 is an example of an exemplary appearance of an LED pad;

FIG. 3 is another example of an exemplary appearance of an LED pad;

FIG. 4 is an exploded view of components of the LED pad shown in FIG. 2;

FIG. 5 is an exploded view of components of the LED pad shown in FIG. 3;

FIG. 6 is an exemplary upper silicone case with grooves;

FIG. 7 is a process flow for manufacturing the LED pad;

FIG. 8 is a partial cross sectional view of an exemplary LED pad in accordance with the present invention; and

FIG. 9 is a partial cross sectional view of another exemplary LED pad in accordance with the present invention.

### DESCRIPTION OF REFERENCE NUMERALS

100: LED pad
110: silicone case
111: upper silicone case
112: lower silicone case
112-1: power connector inlet
112-2: edge surface
112-3: LED module housing space
120: LED module
121: flexible PCB
121-1: reflective layer
121-2: heat conductance layer
122: LED
123: power connector
130: adhesive pad
200: controller
300: power adapter

### MODE FOR INVENTION

The aforementioned effects, features and advantages of the present invention will be more apparent from the description stated below in detail with reference to the accompanying drawings. Therefore, those skilled in the art of the present invention will easily implement the idea of the present invention. In addition, it should be noted that specific description about known technology related to the present invention is omitted if it may unnecessarily obscure the gist of the present invention while describing the present invention. Hereafter, with reference to the accompanying drawings, the preferred embodiments of the present invention are described in detail.

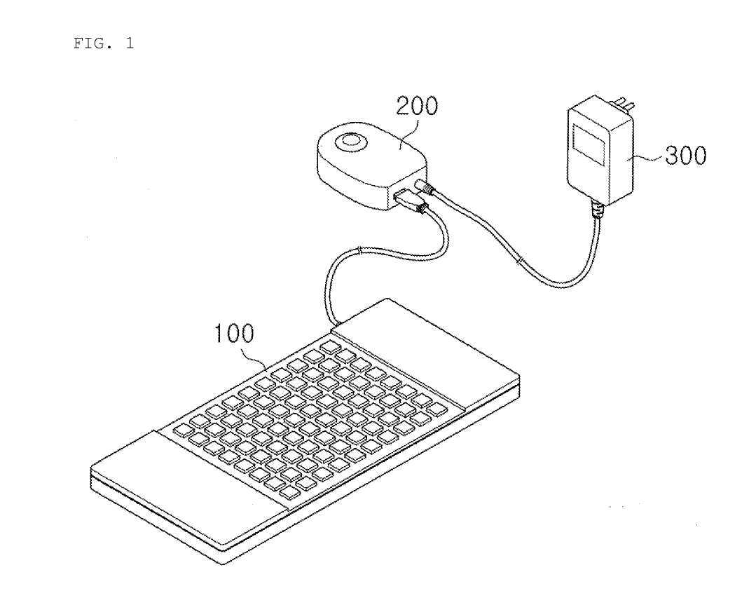

FIG. 1 is an exemplary configuration diagram of a personal treatment apparatus.

The personal treatment apparatus shown in FIG. 1 may include an LED pad 100 and a controller 200, and further include a power adapter 300 for supplying DC power to the controller 200 or the LED pad 100.

Briefly describing each component of the personal treatment apparatus, the LED pad 100 is configured to be connected to the controller 200 and use DC power supplied from the controller 200 to make the LEDs 122 included in the LED pad 100 emit light.

The LED pad 100 is configured to include an LED module 120 which includes a plurality of LEDs 122 to output light therefrom, and a silicone case 110 made of a silicone material for protecting the LED module 120 and insulating it from the outside. Because the main components of the LED pad 100 are made of a bendable material, it may make a close contact easily with user's skin who uses the personal treatment apparatus, and thus may be used with various skin areas for treatment or skin care.

The LED pad 100 is described in detail herein below with reference to the accompanying drawings.

The controller 200 may control the LED pad 100. The controller 200 may include a case and a micro-controller (or processor, hereinafter ‘micom’) mounted on a board included in the case to control the power supplied to the LED pad 100.

The supplied power is sent to the LED pad 100 through a power cable connected between the LED pad 100 and the controller 200, and thus enables the LEDs 122 of the LED pad 100 to be driven.

To this end, the micom may receive input of a button attached on the case (for example, an On/Off button) or a volume switch, supply the DC power received through the power adapter 300 or converted DC power from the DC power to the LED pad 100 depending on the input, or increase/decrease the amount of light of LED 122 as the volume switch controls.

In addition, the micom is configured to have an internal hardware or software timer to prevent user's skin from being exposed excessively to the LED 122 light, and drive the timer after starting to send DC power to stop DC power supply when a predetermined time elapses. The predetermined time may be always fixed (for example, 30 minutes) by the micom, or change by using the button or dip switch attached on the case.

The aforementioned DC power supply and cutoff may be formed by a switch, for example, a transistor mounted on the board included in the controller 200.

The power adapter 300 is connected to a power receptacle to convert AC power to DC power used for the controller 200 or the LED pad 100, and supplies the converted DC power to the controller 200 through a power cable.

The DC power converted as described above is used for driving the LED 122 of the LED pad 100, or the micom of the controller 200, and may be at a specific power level, for example, between 12V and 24V.

The LED pad 100 may be independently used to make a close contact directly with user's skin. Otherwise, the LED pad 100 may be fixed (for example, by using Velcro (fastener), etc.) to a belt configured to be fastened at a specific part of user's body. Such a belt is configured to be fastened at user's body, for example, user's waist, ankle, wrist or thigh, and the LED pad 100 fixed to the belt is configured to make a close contact with a specific part of user's body to emit NIR (Near Infrared Ray) light to the specific part of user's body.

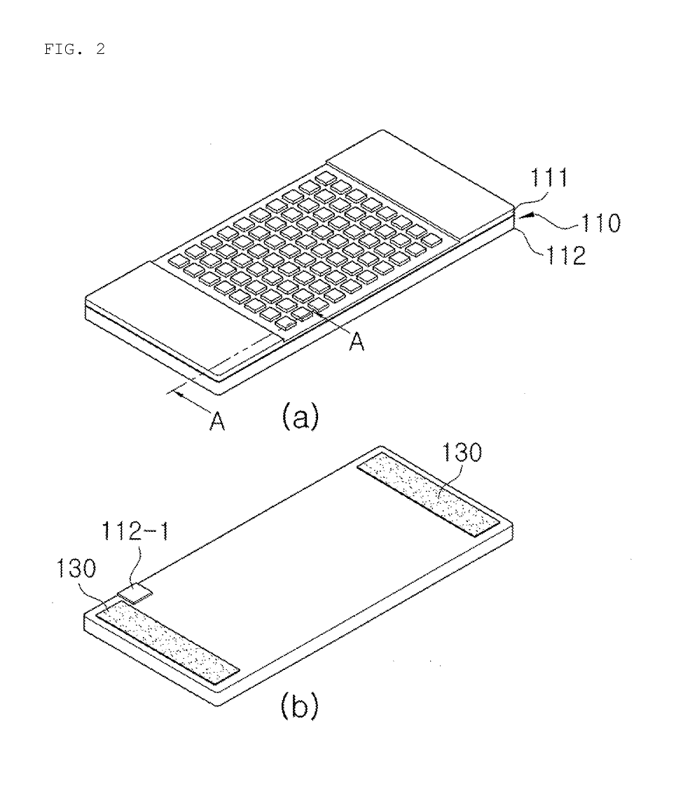

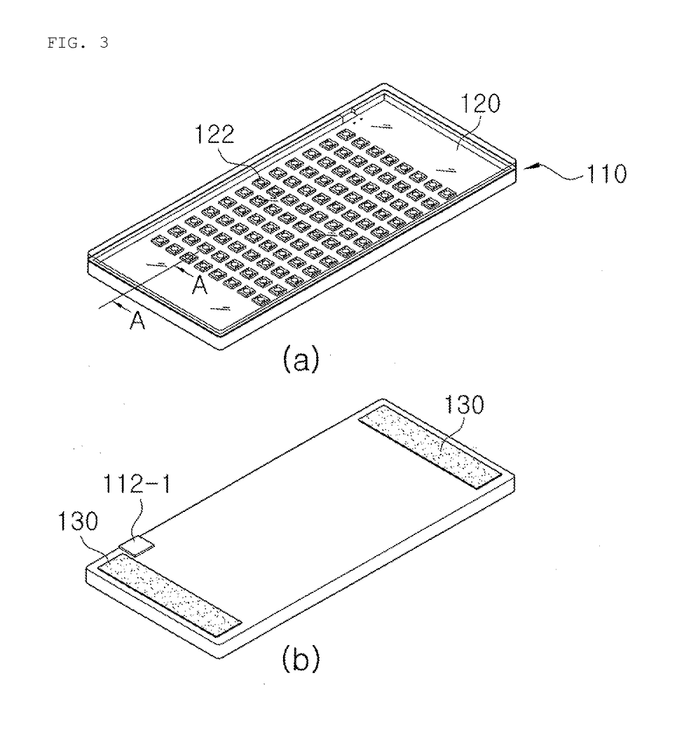

FIG. 2 and FIG. 3 show an exemplary appearance of the LED pad 100, respectively.

FIG. 2 is an exemplary appearance of the LED pad 100 in which the upper silicone case 111 has grooves, and FIG. 3 is another appearance of the LED pad 100 in which the upper silicone case 111 is made of transparent silicone. FIG. 3 is an exemplary appearance without grooves on the upper silicone case 111.

Referring to FIGS. 2 and 3 to describe the LED pad 100, the LED pad 100 is formed with the silicone case 110 for protecting its components therein, insulating them from external impurities or moisture, and forming the shape of the LED pad 100.

The front case of the LED pad 100 (see (A) of FIG. 2 and (A) of FIG. 3) which make a direct contact with a part of user's body is configured to be made of colored or transparent silicone. Preferably, the front case is made of transparent silicone to enhance transmittance of light. If the front case is made of transparent silicone, the LED 122s of the LED pad 100 may be shown from the outside. The rear case is made of transparent or colored silicone.

Two or more adhesive pads 130 are integrated with the rear case of the LED pad 100. The adhesive pads 130 may be so-called Velcro (fastener) tapes. The number of the adhesive pads 130 and the position for integrating them with the rear side of the LED pad 100 may depend on the size of the LED pad 100 or the belt type to which the LED pad 100 is fixed. Therefore, the number may be one, two or more, and their position may be varied.

The LED pad 100 includes an LED module 120 which includes a plurality of LEDs 122 in the silicone case 110. The LED module 120 is configured to be insulated completely from the outside by means of the silicone case 110 integrated as one at the outer side of the LED module 120, and protected within the silicone case 110. Therefore, it is protected fundamentally from incoming external impurity sources.

The rear case is formed with a power connector inlet 112-1 which can accept the power connector 123 to be connected to an external power cable plug.

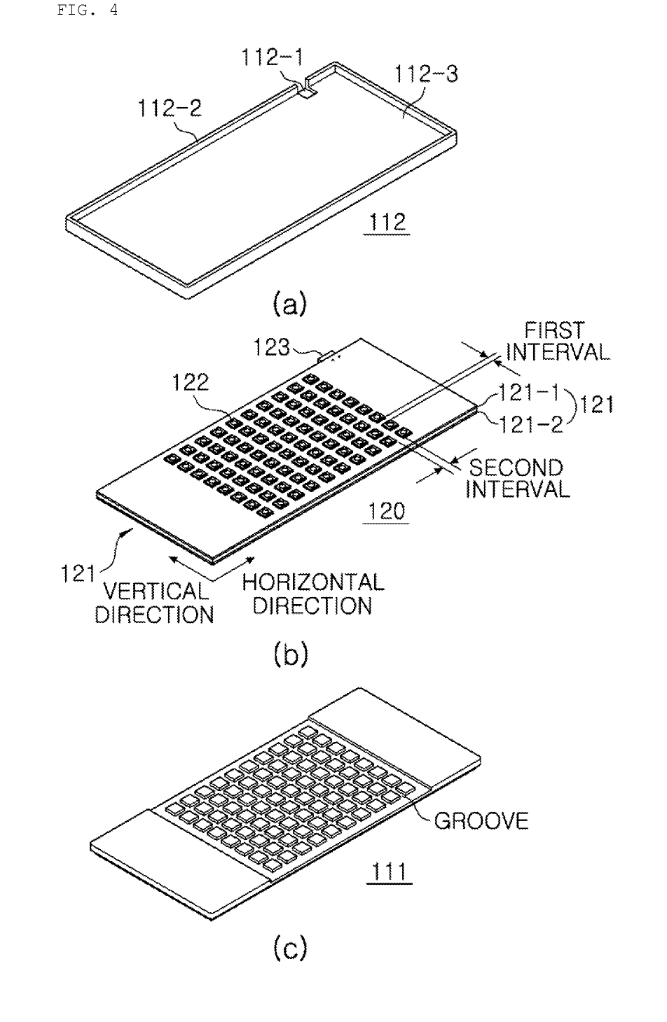

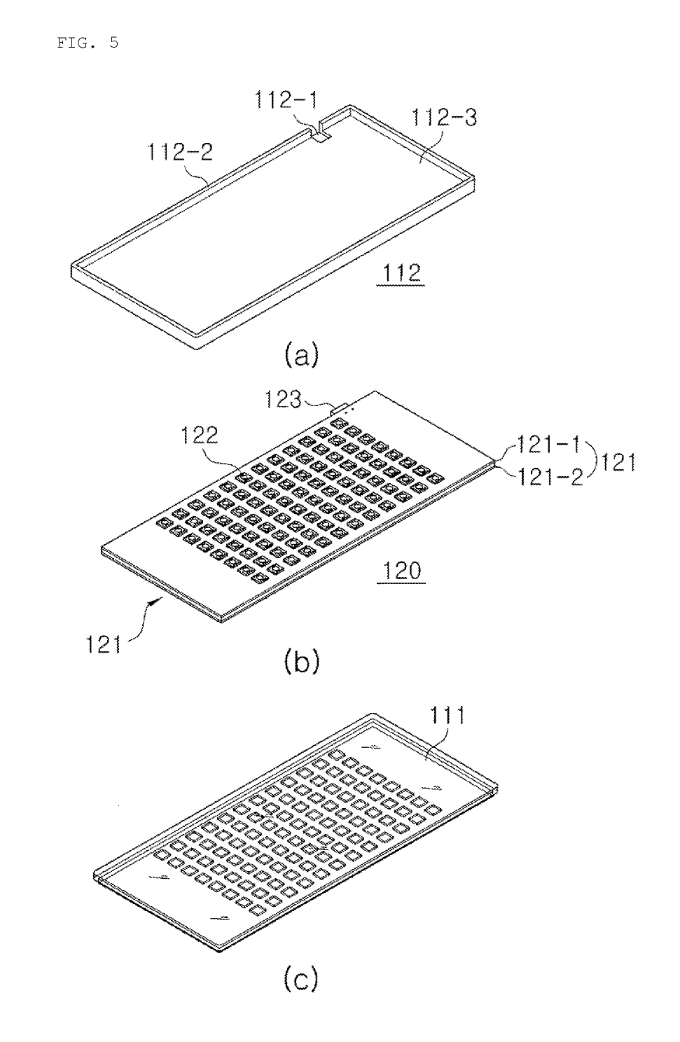

FIGS. 4 and 5 are exploded views of components of the LED pad 100 shown in FIGS. 2 and 3. FIG. 4 shows components of the LED pad 100 shown in FIG. 2, and FIG. 5 shows components of the LED pad 100 shown in FIG. 3.

Referring FIGS. 4 and 5 to describe components of the LED pad 100, the LED pad 100 includes a lower silicone case 112, an upper silicone case 111 integrated with the lower silicone case 112 forming the silicone case 110, and an LED module 120 located in the silicone case 110 composed of an integration of the upper silicone case 111 and the lower silicone case 112.

Preferably, the upper silicone case 111 is transparent, and the lower silicone case 112 is integrated chemically with the upper silicone case 111.

The lower silicone case 112 is made of silicone based material (for example, silicone rubber) to which colored (for example, white) components or transparent components are added. The lower silicone case 112 includes an edge surface 112-2 which is protruded through the outer edge of the lower silicone case 112. The edge surface 112-2 may enable to house the LED module 120 by integration with the power connector inlet 112-1, the upper silicone case 111 and the lower silicone case 112 through injection molding. The lower silicone case 112 also includes an LED module housing space 112-3 which is formed as the internal bottom surface of the lower silicone case 112 along with the edge surface 112-2. The lower silicone case 112 may be pre-molded.

The LED module 120 is configured to include a flexible PCB 121, a plurality of LEDs 122 mounted on or installed in the flexible PCB 121, and a power connector 123 also mounted on or installed in the flexible PCB 121, and to be positioned in the silicone case 110 formed by integrating the upper silicone case 111 with the lower silicone case 112 (preferably, chemical integration by the same molecular components).

Describing each component of the LED module 120 in accordance with the present invention in more detail herein, the flexible PCB 121 includes a heat conductance layer 121-2 facing the internal bottom surface of the lower silicone case 112, and a reflective layer 121-1 positioned on the flexible PCB 121 opposite to the heat conductance layer 121-2 and facing the upper silicone case 111. The flexible PCB 121 may be installed with a plurality of LEDs 122 by SMT (Surface Mounting Technology), etc. Therefore, the heat conductance layer 121-2 is formed on the surface (that is, on the side of lower silicone case 112) opposite to the surface (that is, on the side of upper silicone case 111) on which the plurality of LEDs 122 are installed.

The heat conductance layer 121-2 as described herein is configured to enable to disperse heat radiated following light emission by each of the plurality of LEDs 122 when power is supplied. The heat conductance layer 121-2 may be made of a metallic material with conductivity, for example, silver, copper or aluminum. Preferably, the heat conductance layer 121-2 may be copper foil used in PCB patterning of the flexible PCB 121. Otherwise, the heat conductance layer 121-2 may be attached additionally to the typical flexible PCB 121, where the heat conductance layer 121-2 may be configured to be insulated from the copper foil in PCB patterning of the flexible PCB 121.

The copper foil may send electric signals, and, in addition, a PCB pattern may be formed on the copper foil for sending the electric signals thereto. Therefore, the heat conductance layer 121-2 may also include a PCB pattern for sending power signals supplied from the power connector 123 to each or one of the plurality of LEDs 122.

The copper foil (remaining copper foil not removed from the flexible PCB 121) except the PCB pattern for sending electric signals or the copper foil additionally attached is used for dispersing heat in accordance with the present invention. In addition, the remaining copper foil (or the copper foil additionally attached) not removed from the flexible PCB 121 and dedicated to dispersing heat and the copper foil of the PCB pattern (may also disperse heat) are configured to be insulated.

Because of its metallic feature, the heat conductance layer 121-2 allows heat generated by the light emission by each of the LEDs 122 to be dispersed around the LEDs 122, lowers temperature at the LEDs 122, and keeps the LED pad 100 at constant temperature.

Forming the heat conductance layer 121-2 in the flexible PCB 121 of the LED pad 100 contributes to lowering the temperature at the LEDs 122 which may rise by the light emitted by the LEDs 122, and keeping constant temperature around the LEDs 122 in comparison with conventional LED pads. Therefore, this may enable even more light to be emitted to user's skin which is a close contact with the LED pad (for example, by raising the power level supplied from the power connector 123 or setting the current level as high), user's local skin burns to be prevented, and to maximize the effect of NIR.

The metallic(copper foil) part of the heat conductance layer 121-2 dedicated to dispersing heat is configured on the area corresponding to an area of two or more LEDs 122 to be equivalent to or bigger than the area for the two or more LEDs 122 mounted on the flexible PCB 121. For example, if two LEDs 122 are spaced apart by lcm and each LED 122 size is 0.5 cm×0.5 cm, the heat conductance layer 121-2 for dispersing heat is configured to be at least 1.5 cm×0.5 cm to disperse heat from the LEDs 122 around them, and keep them at constant temperature.

The metallic part of the heat conductance layer 121-2 dedicated to dispersing heat may be arranged preferably on the entire area of the flexible PCB 121 except the PCB pattern that may be included in the heat conductance layer 121-2, and the insulation part of the PCB pattern.

The reflective layer 121-1 is formed on the surface of the flexible PCB 121 opposite to the heat conductance layer 121-2, reflects the light incident through, for example, the transparent upper silicone case 111, and is positioned on the surface of the flexible PCB 121 on the side of the upper silicone case 111.

The reflective layer 121-1 may be, for example, white film or white paint (or ink) having a high light reflectance values. The white film may be glued to the surface of the flexible PCB 121 or the white paint (or ink) may be applied to the surface of flexible PCB 121. The reflective layer 121-1 is formed on, at least, the surface of flexible PCB 121 expect the locations of installed LEDs 122.

With the reflective layer 121-1, NIR light emitted from the LEDs 122 and reflected is reflected again without loss so that NIR light may be used very efficiently.

As described above, the heat conductance layer 121-2 and the reflective layer 121-1 formed on the flexible PCB 121 contribute to enhancing NIR light output, and using the NIR light efficiently. Therefore, since using NIR light is maximized and more NIR light output is implemented with the same power consumption, treatment or the effect of skin care by using NIR is maximized.

The plurality of LEDs 122 included in the LED module 120 and installed in the flexible PCB 121 are the LEDs 122 which emit (or output) the light in a defined range of wavelengths, and may be, for example, an LED package provided by LED manufacturers. The LEDs 122 are an LED package which emits light in the range of wavelengths, for example, NIR, visible light, or ultraviolet rays, and are preferably an LED package which emits NIR.

As seen from FIG. 4 and (B) of FIG. 5 (in particular, see (B) of FIG. 4), the plurality of LEDs 122 are spaced apart at given (defined) intervals and mounted on the flexible PCB 121. The plurality of LEDs 122 is positioned at given (defined) intervals in one direction of the flexible PCB and the other direction perpendicular to the one direction. For example, a plurality of LEDs 122 is positioned at given intervals (hereinafter referred to as ‘first interval’) in the vertical direction of the flexible PCB and a plurality of LEDs 122 is positioned at given intervals (hereinafter referred to as ‘second interval’) in the horizontal direction thereof. The first interval and the second interval may be the same or different depending on design variants.

The power connector 123 included in the LED module 120 is mounted on the flexible PCB 121 to supply power to the plurality of LEDs 122. The power connector 123 includes a power inlet open (or not closed) to house the power cable plug and a contact point to be combined with the plug through the power inlet. The power connector 123 supplies power supplied through the contact point to the plurality of LEDs 122 through, for example, the PCB pattern formed at the heat conductance layer 121-2 or the PCB pattern separately formed.

The power connector 123 may be a standardized connector. For example, the power connector 123 may be a connector defined in the USB (Universal Serial Bus) for sending and receiving power and data, and may be a mini USB connector. The power connector 123 protrudes from an edge of flexible PCB 121 to be mounted, and preferably protrudes by the thickness (for example, 2 mm) of the edge surface 112-2 formed in the lower silicone case 112 to be mounted. Therefore, the power connector 123 and the silicone case 110 may be integrated as one unit and may facilitate integration of the LED module 120 including the power connector 123 with the lower silicone case 112.

Referring again to FIGS. 4 and 5, (C) of FIG. 4 and (C) of FIG. 5 show the upper silicone case 111, respectively.

The upper silicone case 111 is molded through injection molding with liquid silicone, and integrated chemically with the lower silicone case 112. In addition, the upper silicone case 111 is molded by filling the space along the edge of the components of the LED module 120 (for example, the LEDs 122 or the flexible PCB 121) with liquid silicone to protect them from external impact caused by bending the LED pad 100. The liquid silicone is transparent or colored.

The upper silicone case 111 is integrated chemically with the molecular components of the lower silicone case 112 which includes the same molecular components along the edge surface 112-2 of the lower silicone case 112 to form the silicone case 110 as one unit. The upper silicone case 111 is configured to be preferably transparent for transmission of light.

As described above, the upper silicone case 111 is integrated with the lower silicone case 112 as one unit, may maximize the flexibility of the LED pad 100 along with the flexible PCB 121 of the LED module 120 therein. Since the LED module 120 is insulated by means of the silicone case 110, the LED module 120 is protected from contaminations or moisture, and the issues of short circuits or disconnection of the LED module 120 caused by external impact may be further reduced.

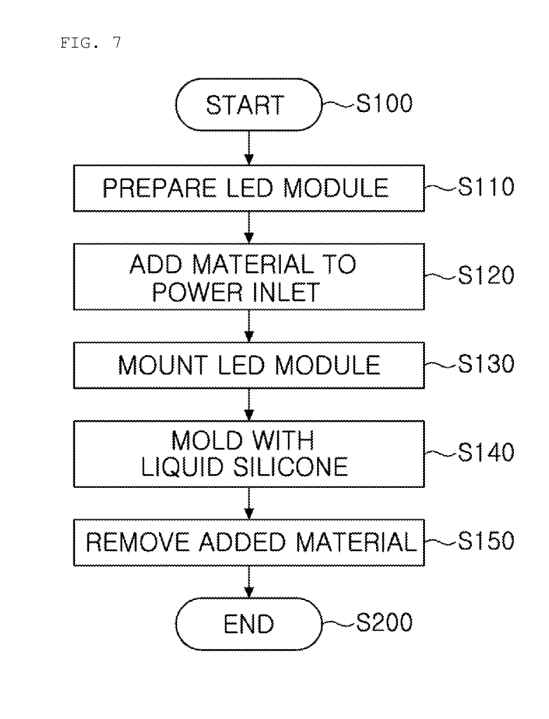

The upper silicone case 111 is molded with the lower silicone case 112 mounted with the LED module 120 through injection molding by using a molding machine, and the process of manufacturing is further described below in detail while describing FIG. 7.

Meanwhile, the upper silicone case 111 is configured to make a close contact with user's body, and LED light is output onto the skin of user's body through the upper silicone case 111. The upper silicone case 111 may have grooves as seen from (C) of FIG. 4. The grooves are positioned between one LED 122 of the plurality of LEDs 122 and another LED 122 adjacent to the one LED 122.

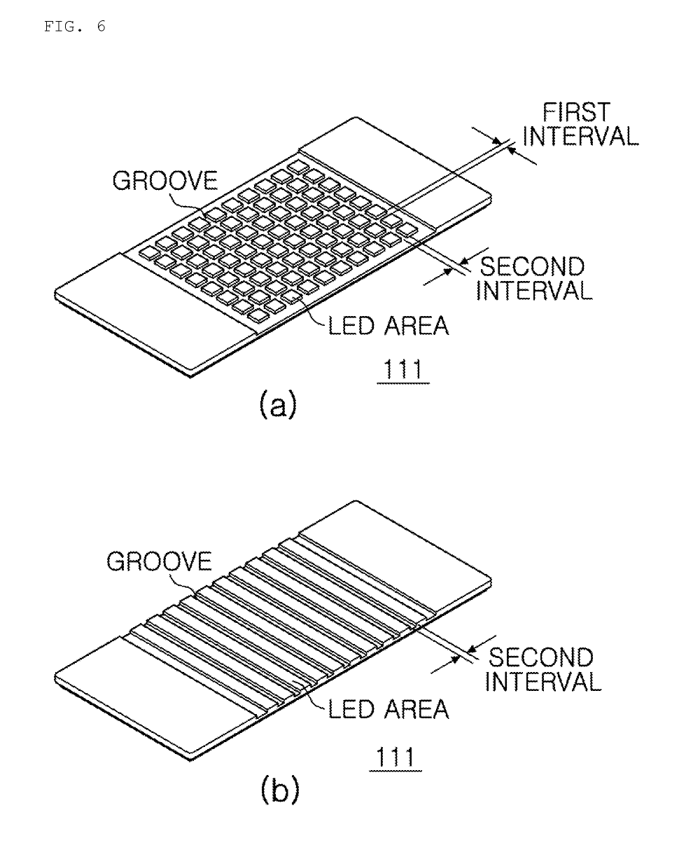

The grooves included in the upper silicone case 111 may be molded in various shapes. FIG. 6 is an exemplary upper silicone case with grooves. (A) of FIG. 6 shows exemplary grooves of which the entire shape is configured as a grid, and (B) of FIG. 6 shows another exemplary grooves of which the entire shape is configured as ditches in one direction.

As seen from (A) of FIG. 6, there are grooves between areas where each of the plurality of LEDs 122 is positioned. The upper silicone case 111 shown in (A) of FIG. 6 has exemplary grooves configured between LEDs 122 adjacent to each of the plurality of LEDs 122 in the vertical and the horizontal directions of the flexible PCB 121 (or the LED pad 100). In addition, the upper silicone case 111 shown in (B) of FIG. 6 has exemplary grooves configured between LEDs 122 adjacent to each of the plurality of LEDs 122 in one direction (horizontal direction) of the flexible PCB 121.

As seen from (A) and (B) of FIG. 6, the upper silicone case 111 makes a close contact with user's body, that is, user's skin directly, and the LED pad 100 provides a path for releasing heat generated following light emission of the LEDs 122 through the grooves included in the upper silicone case 111. The structure of the aforementioned upper silicone case 111 provides a path to allow heat of the LEDs 122 that may be transferred directly to user's skin to be released while making a close contact with user's body, that is, skin. With this structure, users may experience less burning sensation, and more light may be emitted than conventional known personal treatment apparatuses.

FIG. 7 is a process flow for manufacturing the LED pad 100.

For manufacturing the LED pad 100, an LED module 120 is prepared at operation S110. The operation of preparing the LED module may be implemented by manufacturing a flexible PCB 121 including the heat conductance layer 121-2 and the reflective layer 121-1 and configured with a circuit, and installing the plurality of LEDs 122 and a power connector 123 on the flexible PCB 121.

The power connector 123 included in the LED module 120 may include different holes depending on the type or structure of the power connector 123 or the power inlet open toward the outside to house a power cable plug. In such a power connector, it is necessary to protect the power inlet or the holes from liquid silicon entering them in injection molding. Existence of liquid silicone in the power inlet or holes may cause the power connector 123 not to function normally, and thus lower the quality of manufactured goods.

To this end, at operation S120, a particular material is affixed to the power inlet or other open areas (holes, etc.) of the power connector 123 to prevent liquid silicone (for example, transparent liquid silicone; following description is based on the assumption of transparent liquid silicone) from entering or flowing into the power connector 123.

For example, preventing transparent liquid silicone from entering the holes when molding the holes may be implemented by inserting highly adhesive silicone, adhesive tape or a particular material for the purpose into them or gluing it. Preventing transparent liquid silicone from entering the power inlet may be implemented by inserting a plug-shaped material (metallic material (for example, brass alloy) pre-injected as a power cable plug shape, or a material not transformed in molding with transparent liquid silicone) into the power inlet.

Subsequently at operation S130, the LED module 120 is mounted on the transparent or colored (for example, white or orange) lower silicone case 112 pre-molded and manufactured by using the power connector inlet 112-1 and the LED module housing space 112-3. The LED module 120 includes the plurality of LEDs 122, the power connector 123, and the flexible PCB 121 mounted with the plurality of LEDs 122 and the power connector 123. The process of mounting them is facilitated by using the power connector 123 which protrudes as far as the thickness of edge surface 112-2 of the lower silicone case 112 from the edge of the flexible PCB 121.

The operation S130 may be carried out on a molding machine for injection molding.

Subsequently at operation S140, the upper silicone case 111 is molded by putting transparent liquid silicone into the lower silicon case 112 mounted with the LED module 120.

Both the lower silicone case 112 and the upper silicone case 111 are made of silicone. At operation S140, the areas of lower silicone case 112 (for example, the edge surface 112-2, etc.) exposed to the transparent liquid silicone are chemically integrated by using the same molecular components as the transparent liquid silicone to form the silicone case 110 of high integration capacity as one unit and not easily separated.

Therefore, the operation at operation S140 implements the transparent liquid silicone chemically integrated along at least the edge surface 112-2 of the lower silicone case 112, and the transparent liquid silicone put into the flexible PCB 121 of the LED module 120 and the plurality of LEDs 122 may further function as a filler or buffer to mitigate external impact.

Molding with the transparent liquid silicone at operation S140 is completed through curing, and the LED module 120 in the silicon case 110 is completely insulated from the outside and sealed. In addition, the upper silicone case 111 may be molded to include the grooves shown in (A) or (B) of FIG. 6, or not to include any groove.

Subsequently at operation S150, when curing finishes and molding is thus completed by using a molding machine, the material added to the power inlet is removed to complete the process of manufacturing the LED pad 100.

Alternatively, the material to be affixed to the holes of the power connector may not be removed depending on the features of the added material.

The LED pad 100 manufactured as described above is configured so that the silicone case 110 may protect the entire LED module 120 completely from the outside, and the silicone case 110 may be formed through chemical integration to provide an LED pad 100 with various benefits described above to its users.

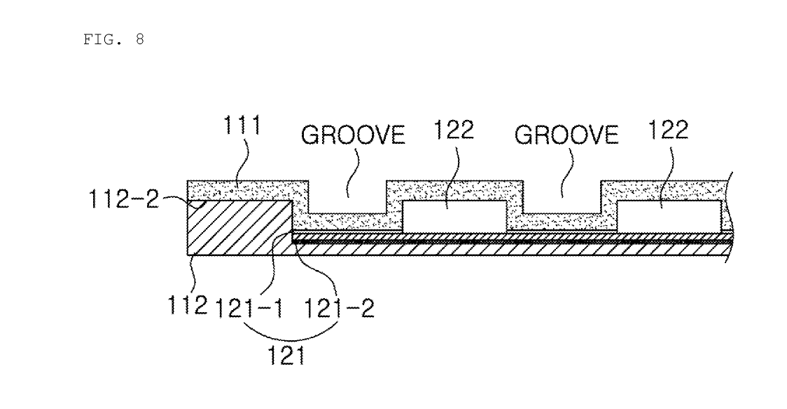

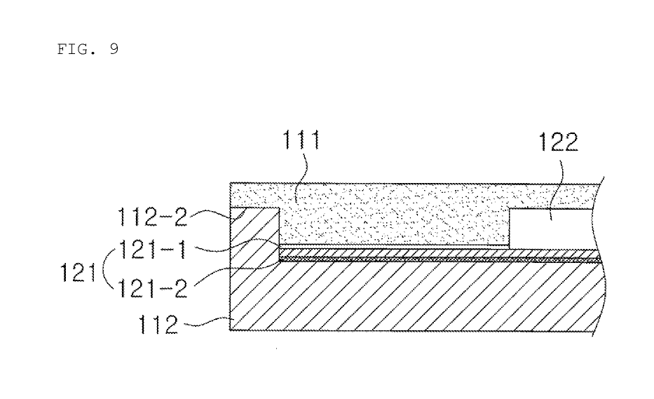

FIGS. 8 and 9 show partial cross sections of the LED pad 100 in accordance with the present invention. FIG. 8 is an exemplary cross sectional view (cross sectional view cut along the line A-A in FIG. 2) with grooves in the upper silicone case 111 of the LED pad 100. FIG. 9 is an exemplary cross section (cross section along the line A-A in FIG. 3) without grooves in the upper silicone case 111 of the LED pad 100. Those cross sections are made by cutting the upper and the lower silicone cases 111 and 112 in the direction from the front of the upper silicone case 111 towards the back of lower silicone case 112.

As seen from FIGS. 8 and 9, the edge surface 112-2 of the lower silicone case 112 and the plane of the lower silicone case 112 extending from the edge surface 112-2 to form the LED module housing space 112-3 are exposed to the transparent liquid silicone in molding by using a molding machine, and chemically integrated with the liquid silicone to enable the silicone on the outer side of the LED module 120 to form one case.

In addition, the space between LEDs 122 on the flexible PCB 121 or between the LEDs 122 and the edge surface 112-2 is configured to be filled with liquid silicone of the upper silicone case 111 to function as a filler or buffer.

As a result, a robust personal treatment apparatus may be provided, which may maximize the flexibility of the LED pad 100, prevent external impurities from entering the pad 100 and mitigate impact.

In addition, as seen from FIG. 8, the upper silicone case 111 of the LED pad 100 is molded to have grooves between the LEDs 122.

In order to provide a more efficient heat release structure, the distance (thickness) from the flexible PCB 121 of the LED pad 100 to the surface of the upper silicone case 111 is configured to be longer (thicker) than the distance from the flexible PCB 121 to the lower silicone case 112.

For example, the thickness from the flexible PCB 121 to the upper silicone case 111 may be configured to range from 3 to 4 mm, and the thickness from the flexible PCB 121 to the lower silicone case 112 may be configured to range from 1 to 2 mm.

The configuration of the LED pad 100 described above enables more heat to be released towards the side opposite to user's skin than the heat released to the skin to implement efficient LED light emission and heat radiation. Furthermore, the LED pad 100 may release more heat radiated by the plurality of LEDs to the lower silicone case 112 through the heat conductance layer 121-2 of the flexible PCB 121 than the heat released to the upper silicone case 111.

### INDUSTRIAL APPLICABILITY

Those skilled in the art of the present invention may easily change, modify or replace the present invention described above without departing from the technical scope of the present invention. Therefore, it should be noted that the present invention is not limited to the embodiments described above and accompanying drawings.

1. An LED pad comprising:
a lower silicone case;
an upper silicon case integrated with the lower silicone case; and
an LED module comprising a plurality of LEDs and a flexible PCB mounted with the plurality of LEDs and positioned in a silicone case formed by integrating the lower silicone case with the upper silicone case.

2. The LED pad of claim 1, wherein the flexible PCB of the LED module comprises:
a reflective layer configured to reflect incident light through the upper silicone case and being positioned on the surface of the flexible PCB; and
a heat conductance layer configured to disperse heat radiated by the plurality of LEDs and formed on the surface opposite to the surface where the plurality of LEDs is mounted.

3. The LED pad of claim 2, wherein the LED module further comprises a power connector configured to supply power to the plurality of LEDs and being mounted on the flexible PCB; and
wherein the power connector supplies power to the plurality of LEDs through a PCB pattern formed in the heat conductance layer and is mounted to protrude as far as the thickness of the edge surface of the lower silicone case.

4. The LED pad of claim 2, wherein the pad further comprises one or more adhesive pads integrated with the lower silicone case and for fixing the LED pad,
wherein the upper silicone case being transparent is integrated chemically with the lower silicone case along the edge of the lower silicone case; the heat conductance layer is copper foil; and the plurality of LEDs are LEDs emitting Near-Infrared Ray.

5. The LED pad of claim 1, wherein the upper silicone case comprises grooves positioned between one LED and other LEDs adjacent to the one LED.

6. The LED pad of claim 5, wherein each of the plurality of LEDs is positioned on the flexible PCB to be spaced apart from other LEDs adjacent to itself in one direction at defined intervals, and other LEDs adjacent to itself in a different direction from the one direction at defined intervals; and
wherein the grooves of the upper silicone case are those positioned only at the one direction or those positioned at the one direction and the different direction.

7. The LED pad of claim 2, wherein the distance from the flexible PCB to the surface of the upper silicone case is configured to be longer than the distance from the flexible PCB to the surface of the lower silicone case; and
wherein the LED pad releases heat radiated by the plurality of LEDs to the lower silicone case through the heat conductance layer.

8. A method for manufacturing an LED pad, the method comprising:
(a) mounting an LED module comprising a plurality of LEDs and a flexible PCB mounted with the plurality of LEDs; and
(b) molding an upper silicone case to be integrated with a lower silicone case with transparent liquid silicone along the edge of the lower silicone case.

9. The method of claim 8, wherein the LED module further comprises a power connector supplying power to the plurality of LEDs, being mounted on the flexible PCB and protruding as far as the edge thickness from the flexible PCB, and
wherein the method for manufacturing an LED pad further comprises:
adding a material to a power inlet of the power connector for preventing the transparent liquid silicone from entering the power inlet before said (a) mounting an LED module; and
removing the added material after said (b) molding an upper silicone case.

10. A personal treatment apparatus comprising an LED pad, the apparatus comprising:
an LED pad as recited in claim 1; and
a controller for controlling power supplied to a plurality of LEDs of the LED pad.
